(19) United States
(12) Patent Application Publication
(10) Pub. No.: US 2019/0314388 A1
(43) Pub. Date: Oct. 17, 2019

(54) PHARMACEUTICAL COMPOSITION CONTAINING DEOXYCHOLIC ACID

(71) Applicant: Penmix Ltd., Chungcheongnam-do (KR)

(72) Inventors: Dongkyu Park, Seoul (KR); Sang Yun Lee, Gyeonggi-do (KR); Young Sub Song, Seoul (KR); Surin Kim, Gyeonggi-do (KR); Seung Jun Lee, Gyeongsangnam-do (KR); Hana Lee, Gyeonggi-do (KR); Seung Ho Ji, Gyeonggi-do (KR)

(21) Appl. No.: 16/384,703

(22) Filed: Apr. 15, 2019

(30) Foreign Application Priority Data
Apr. 16, 2018 (KR) 1020180044131

(51) Int. Cl. A61K 31/575; A61K 47/18; A61K 47/10; A61K 45/06

(52) U.S. Cl. CPC A61K 31/575; A61K 45/06; A61K 47/10; A61K 47/186

(57) ABSTRACT

The present invention provides a lipolysis composition comprising deoxycholic acid or pharmaceutically acceptable salts thereof and a pharmaceutical composition for preventing or treating obesity, comprising, for example, topical obesity. The pharmaceutical composition of the present invention does not cause a precipitate even at a low pH compared to a conventional DCA-containing preparation, thus not only showing excellent storage stability, but also minimizing side effects such as a burning sensation, inflammations, etc. in a site of administration.

### CROSS REFERENCE TO RELATED APPLICATIONS

This application claims all the benefits of priority from Korean Patent Application No. 10-2018-0044131 filed on Apr. 16, 2018 to the Korean Intellectual Property Office; the disclosure of which is incorporated herein in its entirety.

### FIELD

The present invention relates to a pharmaceutical composition comprising deoxycholic acid or pharmaceutically acceptable salts thereof with improved stability. Particularly, the present invention relates to the pharmaceutical composition without causing precipitation at a low pH, but with a reduced side effect.

### BACKGROUND

Fat is one that is accumulated in white adipose tissues as excess energy taken from food, and a state caused by an excessive accumulation of the white adipose tissues conventionally refers to obesity. A process, in which neutral fat is decomposed into free fatty acid (FFA) and glycerol by means of an action of a hormone sensitive lipase (HSL), refers to lipolysis.

To ameliorate such obesity, there are surgical and non-surgical treatment methods.

As the surgical treatment, a liposuction has been representatively performed, but has raised concerns about causing serious side effects including an injury, edema, paralysis and burning sensation in a surgical site, a risk of infection, a damage to skin or nerves, or a perforating injury in vital organs; needs a period for treatment/recovery; and requires a topical anesthesia or a general anesthesia in some cases during a surgical procedure, and thus, an injection therapy, which is the non-surgical treatment, has been preferred. Out of various injection components used in the injection therapy, if water-soluble solutions of sodium deoxycholate (hereinafter referred to as DCA) were injected into fatty tissues in vivo, it is reported that such solutions have properties to remove fat through a cytolysis mechanism (International Patent Publication Nos. WO 2005/117900 and WO 2005/112942, and U.S. Patent Publication Nos. US2005/0261258; US2005/0267080; US2006/127468; and US2006/0154906).

Also, the DCA has an advantage of being easily formulated into a preparation thanks to its high solubility. However, the water-soluble composition of the DCA, in spite of such advantage, in water-soluble solutions selectively including benzyl alcohol, when they are stored at a low concentration for a certain period of time, such compositions are known to form a white precipitate, and surprisingly, it is found that a velocity of precipitation becomes much faster at a lower DCA concentration despite any significant change in pH of solutions. It is known that such precipitation occurs because the hypodermic injections of the DCA are counter-indicated at a low concentration, and considering that cosmetic injections commercially available on the market are valid for 24 months or more on average, an occurrence of precipitates makes it impossible to commercialize those injections for a reason of safety in reality.

To solve such drawbacks, a commercial product without causing a precipitate with pH adjusted up to a higher range of 8.5 has been sold on the market, but reportedly causing side effects such as a burning sensation and inflammations due to a high pH, and thus, there is a need for developing a preparation, which satisfies precipitation stability at a lower pH and has reduced side effects.

### SUMMARY

The present inventors have conducted various researches in order to develop a lipolysis composition without causing a side effect, while showing an excellent lipolysis activity and preparation stability, for example, a pharmaceutical composition for preventing or treating obesity. The present inventors have evaluated the stability and side effects of deoxycholic acid by adding various additives/carriers thereto, and have found that a preparation containing deoxycholic acid does not cause a precipitate even at a low pH compared to a conventional DCA-containing preparation, thus not only showing excellent storage stability, but also minimizing side effects such as a burning sensation, inflammations, etc. in a site of administration.

Accordingly, an objective of the present invention is to provide a pharmaceutical composition for preventing or treating obesity, containing deoxycholic acid as an effective component.

Also, other objective of the present invention is to provide a method for inducing lipolysis or a method for treating obesity, including administering a therapeutically effective amount of deoxycholic acid into a mammal in need of inducing lipolysis or treating obesity.

Further, another objective of the present invention is to provide a use of deoxycholic acid in preparing a medicament for inducing lipolysis or preventing or treating obesity.

The present invention provides a pharmaceutical composition including deoxycholic acid or pharmaceutically acceptable salts thereof with improved stability.

More particularly, the present invention provides the pharmaceutical composition, which includes deoxycholic acid or pharmaceutically acceptable salts thereof, and pharmaceutically acceptable additives and carriers, and has pH of 6.5 to 8.0 with improved stability. The pharmaceutical composition may have pH of 7.0 to 7.9, more particularly pH of 7.2 to 7.8.

In the present invention, the pharmaceutically acceptable additive may be one or more selected from the group consisting of a pH-adjusting agent, isotonic agent, preservative, antimicrobial agent, antioxidant and buffer.

In the present invention, the pharmaceutically acceptable preservative may be one or more selected from the group consisting of benzalkonium chloride, benzaltonium chloride, phenol, cresol, chlorocresol, chlorobutanol and benzyl alcohol, and preferably may be one or more selected from the group consisting of benzalkonium chloride, benzaltonium chloride and benzyl alcohol.

In the present invention, the pharmaceutical composition may be formulated into an aqueous preparation for parenteral administration.

In the present invention, the pharmaceutical composition may be used for preventing or treating obesity. In the pharmaceutical composition of the present invention, it is preferable that the aqueous pharmaceutical composition includes additives/carriers, excluding benzyl alcohol, with pH of 7.0 to 7.9.

Also, in the pharmaceutical composition of the present invention, it is preferable that the pharmaceutical composition includes benzyl alcohol with pH of 7.2 to 7.8.

The pharmaceutical composition of the present invention may be formulated into an aqueous preparation for parenteral administration, and the preparation for parenteral administration may be a preparation for transdermal administration, a preparation for hypodermic administration, a preparation for intravenous administration, a preparation for intramuscular administration, or a preparation for intraperitoneal administration. Also, the preparation for parenteral administration may be a form of solution or emulsion. The preparation for parenteral administration may include one or more pharmaceutically acceptable additives selected from the group consisting of a pH-adjusting agent, isotonic agent, surfactant, stabilizer, preservative, chelating agent, buffer and freeze-drying stabilizer; and one or more pharmaceutically acceptable carriers selected from the group consisting of oil, organic solvent and aqueous solvent.

In the present invention, the pharmaceutical composition may be formulated into a preparation for parenteral administration in a freeze-dried form by adding the additives or carriers thereto.

The present invention also provides a pharmaceutical composition comprising: a first pharmaceutical composition, which includes deoxycholic acid or pharmaceutically acceptable salts thereof, and pharmaceutically acceptable additives and carriers, and has pH of 8.7 or more with improved stability; and a second pharmaceutical composition including one or more components selected from the group consisting of vitamin, lidocaine, procaine, L-carnitine, minophylline, caffeine, vitamin C, placenta, pentoxifylline, clostridium botulisum toxin, polydeoxyribonucleotide (PDRN), hyalironic acid, thioctic acid, glutathion, glycyrrhizin, fursultiamine and hyaluronidase. Particularly, the first pharmaceutical composition may have pH of 9.0 to 9.5. Also, the first pharmaceutical composition and second pharmaceutical composition may be mixed right before an injection. In the first pharmaceutical composition, the solubility of deoxycholic acid is remarkably improved to achieve excellent precipitation stability. Thus, in one aspect of the present invention, there is provided a pharmaceutical composition, which includes deoxycholic acid or pharmaceutically acceptable salts thereof, and pharmaceutically acceptable additives and carriers and has pH of 8.7 or more with improved stability. According to other aspect of the present invention, there is provided a cosmetic composition, which includes deoxycholic acid or cosmetically acceptable salts thereof, and cosmetically acceptable additives and carriers, and has pH of 6.5 to 8.0 with improved stability. According to another aspect of the present invention, there is provided a method for inducing lipolysis or a method for treating obesity, including administering a therapeutically effective amount of deoxycholic acid or pharmaceutically acceptable salts thereof into a mammal in need of inducing lipolysis or treating obesity.

According to another aspect of the present invention, there is provided a use of deoxycholic acid or pharmaceutically acceptable salts thereof in preparing a medicament for inducing lipolysis or preventing or treating obesity.

The pharmaceutical composition of the present invention comprising deoxycholic acid or pharmaceutically acceptable salts thereof shows excellent storage stability, which does not cause a precipitate compared to a conventional DCA-containing preparation. Also, the pharmaceutical composition of the present invention may minimize side effects such as a burning sensation, inflammations, etc., in a site of administration. Accordingly, the pharmaceutical composition of the present invention may significantly secure stability for a storage period above a permissible reference value specified by each of administrative authorities, and thus may be valuably used for preventing or treating obesity, in particular, topical obesity.

### BRIEF DESCRIPTION OF THE DRAWINGS

FIG. 1 shows results of comparatively observing a production of precipitates from preparations at each pH (7.2, 7.4, 7.6, 7.8 and 9.0) of Example 1-1 of the present invention under long-term and accelerated storage conditions for initial, one, three and six months.

FIG. 2 shows results of comparatively observing a production of precipitates from preparations at each pH (7.2, 7.4, 7.6, 7.8 and 9.0) of Example 1-2 of the present invention under long-term and accelerated storage conditions for initial, one, three and six months.

FIG. 3 shows results of comparatively observing a production of precipitates from preparations at each pH (7.2, 7.4, 7.6, 7.8 and 9.0) of Example 2-1 of the present invention under long-term and accelerated storage conditions for initial, one, three and six months.

FIG. 4 shows results of comparatively observing a production of precipitates from preparations at each pH (7.2, 7.4, 7.6, 7.8 and 9.0) of Example 2-2 of the present invention under long-term and accelerated storage conditions for initial, one, three and six months.

FIG. 5 shows results of comparatively observing a production of precipitates from preparations at each pH (7.4, 7.6, 7.8 and 9.0) of Example 3 of the present invention under long-term and accelerated storage conditions for initial, one, three and six months.

FIG. 6 shows results of comparatively observing a production of precipitates from a preparation (pH 8.3) of Comparative Example 2 of the present invention under long-term and accelerated storage conditions for initial, one, three and six months.

FIG. 7 shows results of comparatively observing a production of precipitates from preparations at each pH (7.2, 7.4, 7.6, 7.8 and 9.0) of Comparative Example 1 of the present invention under long-term and accelerated storage conditions for initial, one, three and six months.

FIG. 8 shows results of identifying a content of deoxycholic acid.

### DETAILED DESCRIPTION

The present invention may be variously modified and take on various forms, and thus the exemplary embodiments will be described herein in more detail. However, this is not intended to limit the present invention to a certain disclosure form, but should be construed to include all the modifications, equivalents or substitutes within the spirit and technical scope of the present invention.

Terms used in the present invention are intended to describe a certain exemplary embodiment of the present invention, and the present invention is not limited to the terms.

Terms such as first, second, etc., may be used to describe various components, but the components shall not be limited to the terms. The terms are used only for a purpose of distinguishing one component from the other component. Terms used in the present application are used only to describe a certain exemplary embodiment and are not intended to limit the present invention. Singular forms are to include plural forms, unless otherwise clearly indicated by context. In the present application, terms such as “include(comprise),” “consist of” or the like should be intended to designate a presence of features, numbers, steps, operations, components, parts or combinations thereof described herein, and should not be construed to exclude a possible presence or addition of one or more other features, numbers, steps, operations, components, parts or combinations thereof in advance.

All the terms used herein including technical or scientific terms have the same meaning as commonly understood by those ordinary skilled in the art, to which the present invention pertains, unless defined otherwise. Such terms as those defined in a generally used dictionary are to be interpreted to have the meanings equal to the contextual meanings in the relevant art, and are not to be interpreted to have ideal or excessively formal meanings, unless clearly defined in the present application.

Hereinafter, the present invention will be described in more detail with reference to the accompanying drawings.

According to the present invention, deoxycholic acid has such excellent storage stability as not to cause a precipitate compared to a conventional DCA-containing preparation and minimizes side effects such as a burning sensation, inflammations, etc., at the same time, and thus may be usefully applied to the prevention or treatment of obesity, in particular, topical obesity. Thus, the present invention provides a pharmaceutical composition or a cosmetic composition for preventing, ameliorating or treating obesity, including deoxycholic acid as an effective component.

Deoxycholic acid or salts thereof are all known substances, which may be prepared according to a known method. For example, deoxycholic acid or salts thereof may be prepared into deoxycholic acid or salts thereof with high purity by extracting animal-derived one, e.g., bovine bile acid, and then crystallizing and purifying the resulting extract. Also, the deoxycholic acid or salts thereof may be commercially purchased (for example, available from PCA S.P.A).

Deoxycholic acid of the present invention may be a form of pharmaceutically acceptable salts. Deoxycholic acid is (4R)-4-((3R,5R,10S,12S,13R,17R)-3,12-dihydroxy-10,13-dimethylhexadecahydro-1H-cyclopenta[a]phenanthrene-17-yl)pentanoate, and the salts may be conventional acid-addition salts, for example, salts derived from inorganic acid such as hydrochloric acid, bromic acid, sulfuric acid, phosphoric acid, nitric acid, carbonic acid, etc.; salts of an organic acid group such as citric acid, acetic acid, lactic acid, tartaric acid, maleic acid, fumaric acid, lactobionic acid, salicylic acid, malonic acid, formic acid, propionic acid, oxalic acid, trifluoroacetic acid, benzoic acid, gluconic acid, methanesulfonic acid, glycolic acid, succinic acid, 4-toluenesulfonic acid, glutamic acid or aspartic acid; and the like, but not limited thereto. Also, the salts include a form of conventional metallic salts, for example, alkaline metallic salts such as lithium, sodium or potassium; alkaline earth metallic salts such as calcium or magnesium salt; or chrome salts.

The pharmaceutical composition of the present invention may further include pharmaceutically acceptable salts, preferably sodium salt, as an effective component in addition to deoxycholic acid or pharmaceutically acceptable salts thereof.

The pharmaceutical composition of the present invention may be preferably parenterally administered, and more preferably parenterally topical-administered. The topical administration includes a topical application under eyes, under chins, under arms, on buttocks, calves, backs, thighs, ankles, abdominal sites, etc. For example, the pharmaceutical composition of the present invention may be formulated into a preparation for parenteral administration (including a preparation for topical administration). The preparation for parenteral administration may be a preparation for transdermal administration, a preparation for hypodermic administration, a preparation for intravenous administration, a preparation for intramuscular administration, or a preparation for intraperitoneal administration. Also, the preparation for parenteral administration may be a preparation for single-dose administration or a preparation for multi-dose administration. The preparation for multi-dose administration may be administered by a volume of about 0.2 ml up to a total of six times at an interval of seven days to one month, and may be prepared in such a way that it is suitable to be administered up to 60 times or less per day. The preparation for parenteral administration may be repeatedly administered into a site distanced at an interval of 0.5 to 2.0 cm, if necessary.

The preparation for parenteral administration includes a form of solution, emulsion, suspension, lyophilization, etc., preferably the form of solution or emulsion. The liquid preparation may be sterile-filtered via a bacterial filter, etc., and then may be filled into an ampule, syringe or vial.

The preparation for parenteral administration in the form of the liquid preparation may be prepared according to a formulation method generally used in the manufacturing pharmacy field by using pharmaceutically acceptable additives and/or carriers. Thus, the pharmaceutical composition of the present invention may include one or more pharmaceutically acceptable additives selected from the group consisting of a pH-adjusting agent, isotonic agent, preservative (or antimicrobial agent), antioxidant and buffer; and one or more pharmaceutically acceptable carriers selected from the group consisting of organic solvent and aqueous solvent.

The pH-adjusting agent includes the pH-adjusting agent conventionally used in a preparation for injection, for example, including diethanolamine, acetic acid, meglumine, sodium citrate, sodium hydroxide, adipic acid, citric acid, hydrochloric acid, lactic acid, sulfuric acid, tartaric acid, phosphoric acid, etc., but not limited thereto. The pharmaceutical composition of the present invention may have a range of pH 6.5 to 8.0, e.g., a range of pH 7.0 to 7.9, and more particularly, a range of pH 7.2 to 7.8. The pharmaceutical composition having such pH range may minimize pains and/or inflammations, if being topically administered. For example, a generally healthy human body maintains pH 7.2-7.8, excluding gastric acid and urine, out of which body fluid such as blood, etc. is generally about pH 7.4 (a range of about pH 7.35-7.45). There may be a sensitive response to even a slight change in such pH value, and for example, a composition with more than pH 8, which is too higher than pH of the body fluid, may cause side effects such as pains like a burning sensation, inflammatory responses, etc., in a site of administration. Thus, it is preferable that the composition to be administered in vivo should have a similar range of pH values to the body fluid. The pharmaceutical composition of the present invention may have a range of pH 6.5 to 8.0, e.g., a range of pH 7.0 to 7.9, and more particularly a range of pH 7.2 to 7.8, and thus may minimize side effects such as pains like the burning sensation, or inflammatory responses, if being administered in vivo.

The isotonic agent includes sugar, sugar alcohol, salts, etc., for example, including glucose, glycerin, sodium chloride, calcium chloride, sodium sulfate, glycerin, propylene glycol, polyethylene glycol (for example, polyethylene glycol with a molecular weight of 1000 or less), dextrose, hydroxypropyl betadex, mannitol, potassium chloride, dextran, ficoll, gelatin, hydroxyethyl starch, etc., but not limited thereto. In one exemplary embodiment, the isotonic agent may be glycerin and/or sodium chloride. The isotonic agent may be used in a suitable amount to provide a physiologically acceptable osmotic pressure.

The preservative includes an antimicrobial agent conventionally used in the manufacturing pharmacy field. The preservative includes benzyl alcohol, glycerin, m-cresol, phenol, benzalkonium chloride, benzaltonium chloride, acacia, albumin, alcohol, alginic acid, ascorbyl palmitate, aspartame, boric acid, citric acid, pentetic acid, sodium acetate, sorbic acid, chlorobutanol, o-cresol, p-cresol, chlorocresol, phenylmercuric nitrate, thimerosal, benzoic acid, cholesterol, etc., but not limited thereto. In one exemplary embodiment, the preservative may be benzalkonium chloride, benzaltonium chloride, phenol, cresol, chlorocresol, chlorobutanol and/or benzyl alcohol.

The antioxidant includes alpha tocopherol, butylated hydroxytoluene, butylated hydroxyanisole, potassium metabisulfite, sodium bisulfite, cysteine, etc.

The buffer includes acid, base, salts, etc., for example, including adipic acid, boric acid, calcium carbonate, calcium hydroxide, calcium lactate, tricalcium phosphate, sodium phosphate, citric acid, maleic acid, malic acid, methionine, glycine, sodium glutamate, sodium acetate, sodium hydrogen carbonate, borax, sodium hydroxide, trisodium citrate, sodium lactate, triethanolamine, anhydrous sodium hydrogenphosphate, etc., but not limited thereto. In one exemplary embodiment, the buffer may be anhydrous sodium hydrogenphosphate and/or citric acid.

The aqueous solvent includes, without limitation, water for injection, sterile distilled water, salt water, aqueous dextrose, aqueous sucrose or the like.

The pharmaceutical composition of the present invention may further include a topical anesthetic for relieving pains caused by an injection of injectable solution, an antihistaminic drug for preventing allergy, a lipolysis promoter for promoting lipolysis, a wrinkle improving agent and a collagen production promoter. As the topical anesthetic, the following may be used: lidocaine and procaine, preferably lidocaine; As the antihistaminic drug, the following may be used: piprinhydrinnate and chiorpheniramine, preferably chlorpheniramine; As the fat oxidation promoter, the following may be used: L-carnitine, hyaluronidase, minophylline, caffeine, thioctic acid, etc; As the skin regeneration promoter, the following may be used: vitamin C, placenta, pentoxifylline, clostridium botulisum toxin, polydeoxyribonucleotide (PDRN), hyalironic acid, glutathion, glycyrrhizin, fursultiamine, etc., but not limited thereto.

The pharmaceutical composition of the present invention also provides a first pharmaceutical composition, which includes deoxycholic acid or pharmaceutically acceptable salts thereof, and pharmaceutically acceptable additives and carriers and has pH of 8.7 or more with improved stability, and which is used by being mixed with the other second pharmaceutical composition right before an injection. The first pharmaceutical composition may have pH of e.g., 9.0 to 9.5. The first pharmaceutical composition may be used as a preparation with excellent storage stability because solubility of deoxycholic acid is remarkably improved and thus an occurrence of precipitation is inhibited.

The pharmaceutical composition of the present invention is conventionally contained in a hermetically-sealed and sterilized plastic or organic container. The container may be supplied in a form of specified capacity such as an ampule, vial, syringe or cartridge, or may be supplied in a form of large capacity such as a bag or bottle for injection.

In the pharmaceutical composition of the present invention, a therapeutically effective amount of the deoxycholic acid or pharmaceutically acceptable salts thereof may be in a range of about 1 mg/kg to about 1,500 mg/kg per day, but may change depending on a patient's age, weight, sensitivity, symptom, a form of a preparation or the like. In one exemplary embodiment, deoxycholic acid or pharmaceutically acceptable salts thereof may be included in a range of 1 to 1,000 mg, preferably 1 to 500 mg per a unit preparation.

In the present invention, there is also provided a cosmetic composition, which includes deoxycholic acid or cosmetically acceptable salts thereof, and cosmetically acceptable additives and carriers, and has pH of 6.5 to 8.0 with improved stability. The cosmetically acceptable salts and cosmetically acceptable additives and carriers are the same as the above-described pharmaceutically acceptable salts and pharmaceutically acceptable additives and carriers.

The present invention also includes a method for inducing lipolysis or a method for treating obesity, including administering a therapeutically effective amount of deoxycholic acid or pharmaceutically acceptable salts thereof into a mammal in need of inducing lipolysis or treating obesity.

The present invention also provides a use of deoxycholic acid or pharmaceutically acceptable salts thereof in preparing a medicament for inducing lipolysis or preventing or treating obesity.

Hereinafter, the present invention will be described in more detail through the following examples and experimental examples. However, the following examples and experimental examples are provided only to illustrate the present invention, but are not construed to limit the present invention.

### EXAMPLE 1

### Preparation for a Solution including Benzalkonium

1-1. Preparation for a Solution including Benzalkonium 0.01%

A preparation in a form of solution was prepared in accordance with components and contents as shown in a following table 1. Contents of table 1 indicate a weight (g) of each component. Benzalkonium chloride 0.01% (w/v) was added into 80-85 ml (about 80-85% of a reference amount) of water for injection of about 25-30° C., and fully stirred and dissolved for about 10 minutes. Sodium chloride and disodium phosphate were added into the resulting solution, and stirred for 20 minutes. Sodium hydroxide was dissolved in the resulting solution, after which deoxycholic acid (available from PCA) was added thereinto, and dissolved by stirring at 300 rpm for about 60 minutes. A pH of the solution was about 10.8-11.6. The pH of the resulting solution was set to pH 7.2 with hydrochloric acid, and adjusted to a final volume of 100 ml with water for injection. The resulting solution was filtered via a membrane filter (PES 0.22 μm filter, Millipore, USA), and filled into a vial. The filled vial was loaded onto a terminal sterilizer (Systec DX-200, Germany), and sterilized at a set temperature of 121° C. for 30 minutes.

Also, the vials were prepared by means of the same method as above, except for adjusting pH to 7.4, 7.6, 7.8 and 9.0 respectively, as shown in the following table 1.

TABLE 1

| | | Example 1-1 | | | | |
|---|---|---|---|---|---|---|
| | | 1 | 2 | 3 | 4 | 5 |
| Solution | Deoxycholic acid | 1.0 | 1.0 | 1.0 | 1.0 | 1.0 |
| | Sodium chloride | 0.44 | 0.44 | 0.44 | 0.44 | 0.44 |
| | Sodium phosphate | 0.14 | 0.14 | 0.14 | 0.14 | 0.14 |
| | dibasic | | | | | |
| | Benzyl alcohol | — | — | — | — | — |
| | Benzalkonium | 0.01 | 0.01 | 0.01 | 0.01 | 0.01 |
| | chloride | | | | | |
| | Sodium hydroxide | 0.14 | 0.14 | 0.14 | 0.14 | 0.14 |
| | Hydrochloric acid | — | — | — | — | — |

| | | | | | |
|---|---|---|---|---|---|
| Water for injection | 100 | 100 | 100 | 100 | 100 |
| (final volume, ml) | | | | | |
| pH range | pH 7.2 | pH 7.4 | pH 7.6 | pH 7.8 | pH 9.0 |

1-2. Preparation for a Solution including Benzalkonium 0.02%

A preparation in a form of solution was prepared in accordance with components and contents as shown in a following table 2. Contents of table 2 indicate a weight (g) of each component. Benzalkonium chloride 0.02% (w/v) was added into 80-85 ml (about 80-85% of a reference amount) of water for injection of about 25-30° C., and fully stirred and dissolved for about 10 minutes. Sodium chloride and disodium phosphate were added into the resulting solution, and stirred for 20 minutes. Sodium hydroxide was dissolved in the resulting solution, after which deoxycholic acid was added thereinto, and dissolved by stirring at 350 rpm for about 30 minutes. A pH of the solution was about 10.8-11.6. The pH of the resulting solution was set to pH 7.2 with hydrochloric acid, and adjusted to a final volume of 100 ml with water for injection. The resulting solution was filtered via a membrane filter (PVDF 0.22 μm filter, Millipore, USA), and filled into a vial. The filled vial was loaded onto a terminal sterilizer (Systec DX-200, Germany), and sterilized at a set temperature of 121° C. for 30 minutes.

Also, the vials were prepared by means of the same method as above, except for adjusting pH to 7.4, 7.6, 7.8 and 9.0 respectively, as shown in the following table 2.

TABLE 2

| | | Example 1-2 | | | | |
|---|---|---|---|---|---|---|
| | | 1 | 2 | 3 | 4 | 5 |
| Solution | Deoxycholic acid | 1.0 | 1.0 | 1.0 | 1.0 | 1.0 |
| | Sodium chloride | 0.44 | 0.44 | 0.44 | 0.44 | 0.44 |
| | Sodium phosphate | 0.14 | 0.14 | 0.14 | 0.14 | 0.14 |
| | dibasic | | | | | |
| | Benzyl alcohol | — | — | — | — | — |
| | Benzalkonium | 0.02 | 0.02 | 0.02 | 0.02 | 0.02 |
| | chloride | | | | | |
| | Sodium hydroxide | 0.14 | 0.14 | 0.14 | 0.14 | 0.14 |
| | Hydrochloric acid | — | — | — | — | — |

| | | | | | |
|---|---|---|---|---|---|
| Water for injection | 100 | 100 | 100 | 100 | 100 |
| (final volume, ml) | | | | | |
| pH range | pH 7.2 | pH 7.4 | pH 7.6 | pH 7.8 | pH 9.0 |

### EXAMPLE 2

### Preparation for a Solution including Benzaltonium

2-1. Preparation for a Solution including Benzaltonium 0.01%

A preparation in a form of solution was prepared in accordance with components and contents as shown in a following table 3. Contents of table 3 indicate a weight (g) of each component. Benzaltonium chloride 0.01% (w/v) was added into 80-85 ml (about 80-85% of a reference amount) of water for injection of about 25-30° C., and fully stirred and dissolved for about 10 minutes. Sodium chloride and disodium phosphate were added into the resulting solution, and stirred for 20 minutes. Sodium hydroxide was dissolved in the resulting solution, after which deoxycholic acid was added thereinto, and dissolved by stirring at 300 rpm for about 60 minutes. A pH of the solution was about 10.8-11.6. The pH of the resulting solution was set to pH 7.2 with hydrochloric acid, and adjusted to a final volume of 100 ml with water for injection. The resulting solution was filtered via a membrane filter (PVDF 0.22 μm filter, Millipore, USA), and filled into a vial. The filled vial was loaded onto a terminal sterilizer (Systec DX-200, Germany), and sterilized at a set temperature of 121° C. for 30 minutes.

Also, the vials were prepared by means of the same method as above, except for adjusting pH to 7.4, 7.6, 7.8 and 9.0 respectively, as shown in the following table 3.

TABLE 3

| | | Example 2-1 | | | | |
|---|---|---|---|---|---|---|
| | | 1 | 2 | 3 | 4 | 5 |
| Solution | Deoxycholic acid | 1.0 | 1.0 | 1.0 | 1.0 | 1.0 |
| | Sodium chloride | 0.44 | 0.44 | 0.44 | 0.44 | 0.44 |
| | Sodium phosphate | 0.14 | 0.14 | 0.14 | 0.14 | 0.14 |
| | dibasic | | | | | |
| | Benzyl alcohol | — | — | — | — | — |
| | Benzaltonium | 0.01 | 0.01 | 0.01 | 0.01 | 0.01 |
| | chloride | | | | | |
| | Sodium hydroxide | 0.14 | 0.14 | 0.14 | 0.14 | 0.14 |
| | Hydrochloric acid | — | — | — | — | — |

| | | | | | |
|---|---|---|---|---|---|
| Water for injection | 100 | 100 | 100 | 100 | 100 |
| (final volume, ml) | | | | | |
| pH range | pH 7.2 | pH 7.4 | pH 7.6 | pH 7.8 | pH 9.0 |

2-2. Preparation for a Solution including benzaltonium 0.02%

A preparation in a form of solution was prepared in accordance with components and contents as shown in a following table 4. Contents of table 4 indicate a weight (g) of each component. Benzaltonium chloride 0.02% (w/v) was added into 80-85 ml (about 80-85% of a reference amount) of water for injection of about 25-30° C., and fully stirred and dissolved for about 10 minutes. Sodium chloride and disodium phosphate were added into the resulting solution, and stirred for 20 minutes. Sodium hydroxide was dissolved in the resulting solution, after which deoxycholic acid was added thereinto, and dissolved by stirring at 350 rpm for about 30 minutes. A pH of the solution was about 10.8-11.6. The pH of the resulting solution was set to pH 7.2 with hydrochloric acid, and adjusted to a final volume of 100 ml with water for injection. The resulting solution was filtered via a membrane filter (PVDF 0.22 μm filter, Millipore, USA), and filled into a vial. The filled vial was loaded onto a terminal sterilizer (Systec DX-200, Germany), and sterilized at a set temperature of 121° C. for 30 minutes.

Also, the vials were prepared by means of the same method as above, except for adjusting pH to 7.4, 7.6, 7.8 and 9.0 respectively, as shown in the following table 4.

TABLE 4

| | | Example 2-2 | | | | |
|---|---|---|---|---|---|---|
| | | 1 | 2 | 3 | 4 | 5 |
| Solution | Deoxycholic acid | 1.0 | 1.0 | 1.0 | 1.0 | 1.0 |
| | Sodium chloride | 0.44 | 0.44 | 0.44 | 0.44 | 0.44 |
| | Sodium phosphate | 0.14 | 0.14 | 0.14 | 0.14 | 0.14 |
| | dibasic | | | | | |
| | Benzyl alcohol | — | — | — | — | — |
| | Benzaltonium | 0.02 | 0.02 | 0.02 | 0.02 | 0.02 |
| | chloride | | | | | |
| | Sodium hydroxide | 0.14 | 0.14 | 0.14 | 0.14 | 0.14 |
| | Hydrochloric acid | — | — | — | — | — |

| | | | | | |
|---|---|---|---|---|---|
| Water for injection | 100 | 100 | 100 | 100 | 100 |
| (final volume, ml) | | | | | |
| pH range | pH 7.2 | pH 7.4 | pH 7.6 | pH 7.8 | pH 9.0 |

### EXAMPLE 3

### Preparation for a Solution including Benzyl alcohol

A preparation in a form of solution was prepared in accordance with components and contents as shown in a following table 5. Contents of table 5 indicate a weight (g) of each component. Benzyl alcohol 0.9% (w/v) was added into 80-85 ml (about 80-85% of a reference amount) of water for injection of about 25-30° C., and fully stirred and dissolved for about 10 minutes. Sodium chloride and disodium phosphate were added into the resulting solution, and stirred for 20 minutes. Sodium hydroxide was dissolved in the resulting solution, after which deoxycholic acid was added thereinto, and dissolved by stirring at 300 rpm for about 60 minutes. A pH of the solution was about 10.8-11.6. The pH of the resulting solution was set to pH 7.4 with hydrochloric acid, and adjusted to a final volume of 100 ml with water for injection. The resulting solution was filtered via a membrane filter (PES 0.22 μm filter, Millipore, USA), and filled into a vial. The filled vial was loaded onto a terminal sterilizer (Systec DX-200, Germany), and sterilized at a set temperature of 121° C. for 30 minutes.

Also, the vials were prepared by means of the same method as above, except for adjusting pH to 7.6, 7.8 and 9.0 respectively, as shown in the following table 5.

TABLE 5

| | | Example 3 | | | |
|---|---|---|---|---|---|
| | | 1 | 2 | 3 | 4 |
| Solution | Deoxycholic acid | 1.0 | 1.0 | 1.0 | 1.0 |
| | Sodium chloride | 0.44 | 0.44 | 0.44 | 0.44 |
| | Sodium phosphate | 0.14 | 0.14 | 0.14 | 0.14 |
| | dibasic | | | | |
| | Benzyl alcohol | 0.9 | 0.9 | 0.9 | 0.9 |
| | Benzaltonium | — | — | — | — |
| | chloride | | | | |
| | Sodium hydroxide | 0.14 | 0.14 | 0.14 | 0.14 |
| | Hydrochloric acid | — | — | — | — |

| | | | | |
|---|---|---|---|---|
| Water for injection | 100 | 100 | 100 | 100 |
| (final volume, ml) | | | | |
| pH range | pH 7.4 | pH 7.6 | pH 7.8 | pH 9.0 |

### COMPARATIVE EXAMPLE 1

### Preparation for a Solution without including any Preservative

A preparation in a form of solution was prepared in accordance with components and contents as shown in a following table 6. Contents of table 6 indicate a weight (g) of each component. Sodium chloride and disodium phosphate were added into 80-85 ml (about 80-85% of a reference amount) of water for injection of about 25-30° C., and stirred and dissolved for about 10 minutes. Sodium hydroxide was dissolved in the resulting solution, after which deoxycholic acid was added thereinto, and dissolved by stirring at 300 rpm for about 60 minutes. At that time, a pH of the solution was about 10.6-11.6. The pH of the resulting solution was set to pH 7.2 with hydrochloric acid, and adjusted to a final volume of 100 ml with water for injection. The resulting solution was filtered via a membrane filter (PES 0.22 μm filter, Millipore, USA), and filled into a vial. The filled vial was loaded onto a terminal sterilizer (Systec DX-200, Germany), and sterilized at a set temperature of 121° C. for 30 minutes.

Also, the vials were prepared by means of the same method as above, except for adjusting pH to 7.4, 7.6, 7.8 and 9.0 respectively, as shown in the following table 6.

TABLE 6

| | | Comparative Example 1 | | | | |
|---|---|---|---|---|---|---|
| | | 1 | 2 | 3 | 4 | 5 |
| Solution | Deoxycholic acid | 1.0 | 1.0 | 1.0 | 1.0 | 1.0 |
| | Sodium chloride | 0.44 | 0.44 | 0.44 | 0.44 | 0.44 |
| | Sodium phosphate | 0.14 | 0.14 | 0.14 | 0.14 | 0.14 |
| | dibasic | | | | | |
| | Benzyl alcohol | — | — | — | — | — |
| | Sodium hydroxide | 0.14 | 0.14 | 0.14 | 0.14 | 0.14 |
| | Hydrochloric acid | — | — | — | — | — |

| | | | | | |
|---|---|---|---|---|---|
| Water for injection | 100 | 100 | 100 | 100 | 100 |
| (final volume, ml) | | | | | |
| pH range | pH 7.2 | pH 7.4 | pH 7.6 | pH 7.8 | pH 9.0 |

### COMPARATIVE EXAMPLE 2

### Preparation for a Commercial Preparation

A preparation of Comparative Example was prepared in accordance with components and contents as shown in a following table 7, with reference to the examples of the Korean Patent No. 10-1751585. Contents of table 7 indicate a weight (g) of each component. Benzyl alcohol was added into 80-85 ml (about 80-85% of a reference amount) of water for injection of about 25-30° C., and fully stirred and dissolved for about 10 minutes or more. Sodium chloride and disodium phosphate were added into the resulting solution, and stirred for 20 minutes. Sodium hydroxide was dissolved in the resulting solution, after which deoxycholic acid was added thereinto, and dissolved by stirring at 300 rpm for about 60 minutes. A pH of the solution was about 10.6-11.6. The pH of the resulting solution was set to pH 8.3 with hydrochloric acid, and adjusted to a final volume of 100 ml with water for injection. The resulting solution was filtered via a membrane filter (PES 0.22 μm filter, Millipore, USA), and filled into a vial. The filled vial was loaded onto a terminal sterilizer (Systec DX-200, Germany), and sterilized at a set temperature of 121° C. for 30 minutes.

TABLE 7

| | | Comparative Example 2 |
|---|---|---|
| Solution | Deoxycholic acid | 1.0 |
| | Sodium chloride | 0.44 |
| | Sodium phosphate dibasic | 0.14 |
| | Benzyl alcohol | 0.9 |
| | Benzalkonium chloride | — |
| | Sodium hydroxide | 0.14 |
| | Hydrochloric acid | — |

| | |
|---|---|
| Water for injection | 100 |
| (final volume, ml) | |
| pH range | pH 8.3 |

### EXPERIMENTAL EXAMPLE 1

### Stability Test

### Experimental Example 1-1

### Stability Test of Example 1

The preparations of Example 1 (Examples 1-1 and 1-2), produced only with a pH range varying from 7.2 to 9.0, were respectively subjected to long-term storage (25° C., humidity 60%) and accelerated storage (40° C., humidity 75%) for zero day (Initial), one, three and six months, and compared with each other.

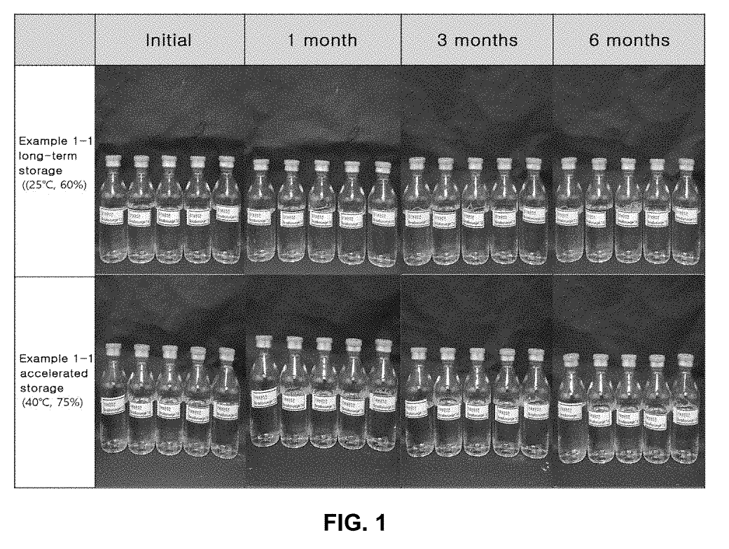

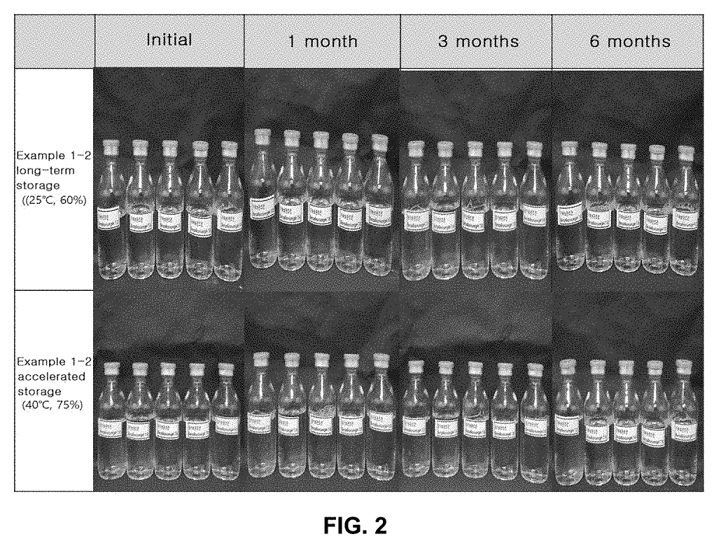

In result, it might be identified that the preparations of Example 1 of the present invention have excellent storage stability without causing a precipitate, as shown in FIGS. 1 and 2.

### Experimental Example 1-2

### Stability Test of Example 2

The preparations of Example 2 (Examples 2-1 and 2-2), produced only with a pH range varying from 7.2 to 9.0, were respectively subjected to long-term storage (25° C., humidity 60%) and accelerated storage (40° C., humidity 75%) for zero day (Initial), one, three and six months, and compared with each other.

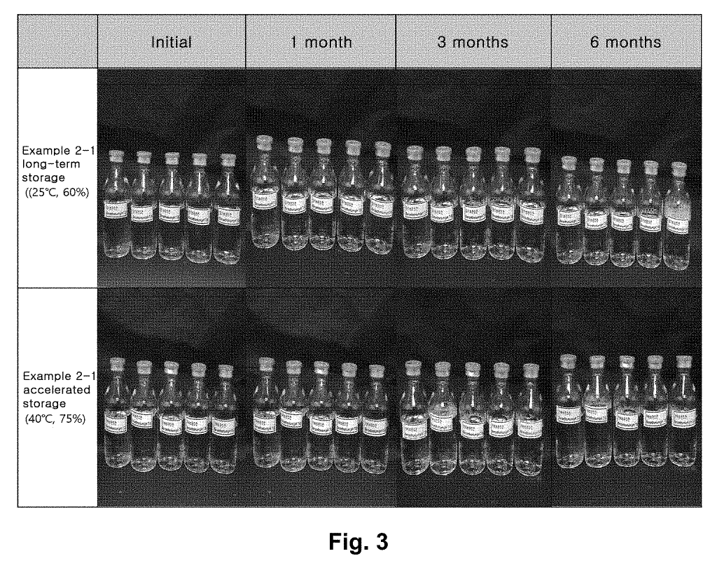

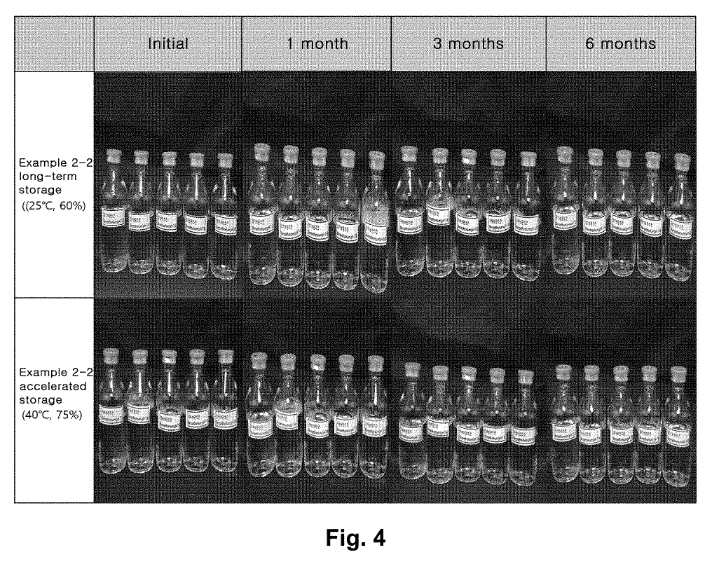

In result, it might be identified that the preparations of Example 2 of the present invention have excellent storage stability without causing a precipitate, as shown in FIGS. 3 and 4.

### Experimental Example 1-3

### Stability Test of Example 3

The preparations of Example 3, produced only with a pH range varying from 7.4 to 9.0, as well as the (commercial) preparations of Comparative Example 2, produced with a pH range adjusted to pH 8.3, were respectively subjected to long-term storage (25° C., humidity 60%) and accelerated storage (40° C., humidity 75%) for zero day (Initial), one, three and six months, and compared with each other.

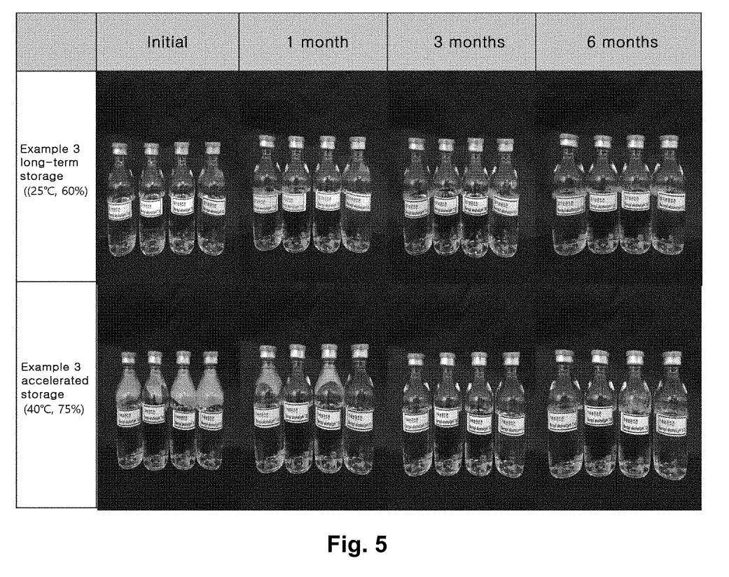

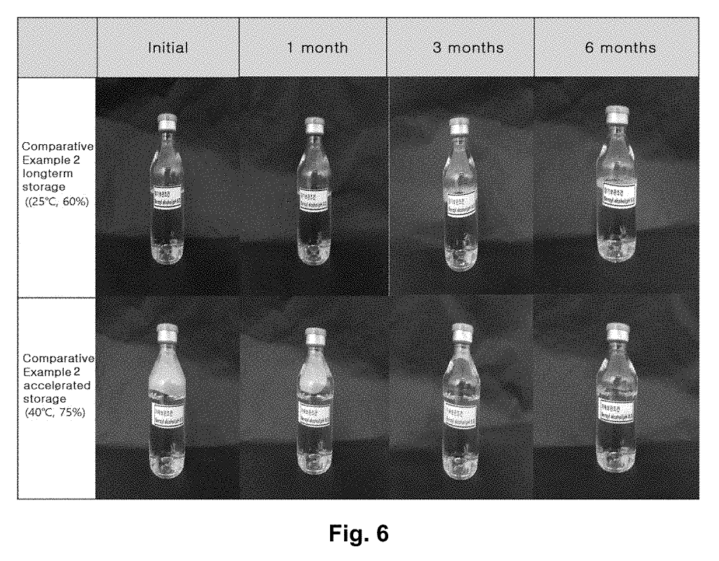

In result, it might be identified that the preparations of Example 3 of the present invention have excellent storage stability without causing a phenomenon of precipitation at the same level as the (commercial) preparations of Comparative Example 2, as shown in FIGS. 5 and 6.

### Experimental Example 1-4.

### Stability Test of Comparative Example 1

The preparations of Comparative Example 1, produced only with a pH range varying from 7.2 to 9.0, were respectively subjected to long-term storage (25° C., humidity 60%) and accelerated storage (40° C., humidity 75%) for zero day (linitial), one, three and six months, and compared with each other.

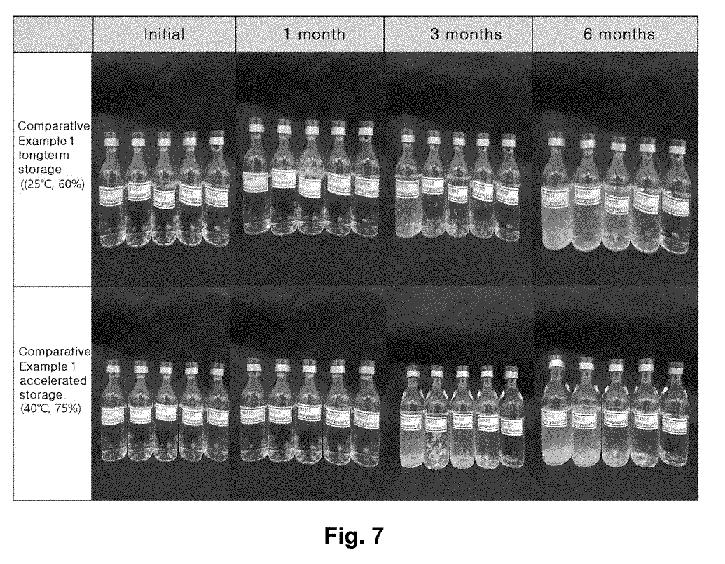

In result, it might be identified with naked eyes that the preparations of Comparative Example 1 cause a phenomenon of precipitation with time and produce a more amount of precipitates with a lower pH, as shown in FIG. 7.

### EXPERIMENTAL EXAMPLE 2

### Test on Content Changes of Examples

In order to identify a content of Examples prepared above, a test was performed by means of a column of InfinityLab poroshell 120 EC-C18 (4.6×150 mm, 4 μm, USA), a high-pressure liquid chromatography (Agilent Technologies 1260 Infinity II HPLC/GPC, Agilent, USA) and an ELSD detector (Agilent Technologies 1260 Infinity II ELSD, Agilent, USA) with reference to analysis conditions as shown in the following table 8. A detection was performed under the condition of a mobile phase (A) with 0.1% formic acid in water; (B) with 0.1% formic acid in ACN; a diluent with methanol/water (80:20); a column temperature of 25° C.; a flow rate of 1.0 ml per minute; an injected amount of 25 μL; an evaporator and nebulizer temperature of 60° C.; and a nitrogen gas flow rate of 1.1 L/min, and a content of Examples was identified by using a sample concentration of 0.1 mg/m L.

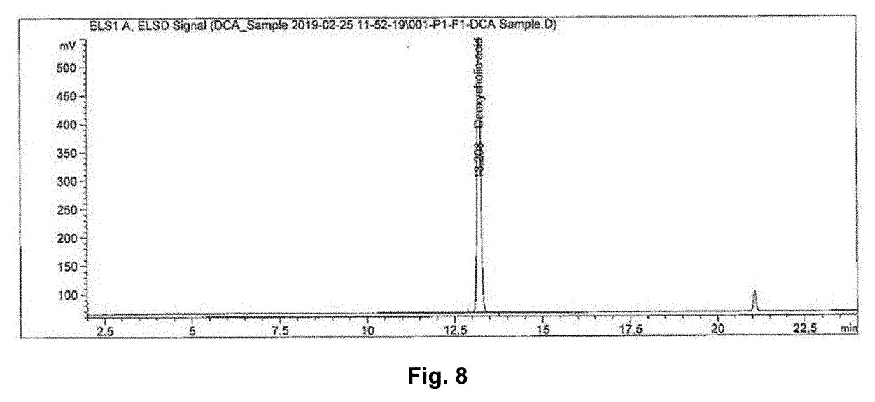

In result, it might be identified that the preparations of Examples appear at 13.2 minutes, as shown in FIG. 8, thus suggesting that there do not occur any deoxycholic acid and content changes.

TABLE 8

| Minute (min) | A % | B % |
|---|---|---|
| 0.0 | 75 | 25 |
| 2.0 | 55 | 45 |
| 14.0 | 42 | 58 |
| 24.0 | 0 | 100 |
| 35.0 | 0 | 100 |
| 38.0 | 75 | 25 |
| 45.0 | 75 | 25 |

### EXPERIMENTAL EXAMPLE 3

### Test on Measurement of Insoluble Minute Particles

A measurement was performed on the insoluble minute particles (HIAC 9703+, MT-C-006) of the compositions of Examples prepared above. Such compositions satisfied the permissible reference values for 6000 or less particles having 10 μm or more in diameter and 600 or less particles having 25 μm or more in diameter per vial. The results of the measurement are as shown in tables 9 and 10, respectively.

In result, it was identified that such compositions contain insoluble minute particles at a level equal to or similar to Comparative 2, with regard to the number of insoluble minute particles having 10 μm or more in size and the number of insoluble minute particles having 25 μm or more in size, as shown in the following tables 9 and 10.

TABLE 9

| | | Evaluation Index (10 μm) | | | | | | | |
|---|---|---|---|---|---|---|---|---|---|
| | | Long-Term | | | | Accelerated | | | |
| | | 0 | 1 | 3 | 6 | 0 | 1 | 3 | 6 |
| Example | | day | month | months | months | day | months | months | months |
| Example 1-1 | 7.2 | 161 | 22 | 21 | 15 | 161 | 27 | 38 | 17 |
| | 7.4 | 18 | 10 | 5 | 1 | 18 | 4 | 12 | 3 |
| | 7.6 | 8 | 5 | 1 | 1 | 8 | 4 | 5 | 1 |
| | 7.8 | 11 | 4 | 4 | 2 | 11 | 2 | 2 | 1 |
| | 9.0 | 23 | 5 | 7 | 8 | 23 | 1 | 3 | 1 |
| Example 1-2 | 7.2 | 157 | 24 | 6 | 1 | 157 | 37 | 7 | 6 |
| | 7.4 | 19 | 12 | 6 | 3 | 19 | 13 | 6 | 2 |
| | 7.6 | 12 | 7 | 5 | 2 | 12 | 5 | 5 | 1 |
| | 7.8 | 19 | 5 | 3 | 2 | 19 | 2 | 2 | 2 |
| | 9.0 | 8 | 3 | 2 | 2 | 8 | 4 | 1 | 1 |
| Example 2-1 | 7.2 | 105 | 11 | 25 | 16 | 105 | 27 | 22 | 17 |
| | 7.4 | 85 | 9 | 22 | 10 | 85 | 12 | 11 | 4 |
| | 7.6 | 36 | 7 | 1 | 1 | 36 | 5 | 1 | 1 |
| | 7.8 | 63 | 15 | 6 | 1 | 63 | 2 | 5 | 1 |
| | 9.0 | 57 | 3 | 2 | 1 | 57 | 2 | 1 | 1 |
| Example 2-2 | 7.2 | 135 | 29 | 25 | 14 | 135 | 22 | 21 | 14 |
| | 7.4 | 48 | 37 | 18 | 12 | 48 | 12 | 9 | 7 |
| | 7.6 | 23 | 8 | 1 | 1 | 23 | 14 | 12 | 1 |
| | 7.8 | 15 | 4 | 3 | 2 | 15 | 0 | 1 | 0 |
| | 9.0 | 7 | 1 | 1 | 1 | 7 | 0 | 1 | 1 |
| Example 3 | 7.4 | 37 | 21 | 10 | 4 | 37 | 22 | 10 | 5 |
| | 7.6 | 36 | 12 | 6 | 3 | 36 | 11 | 10 | 4 |
| | 7.8 | 15 | 7 | 6 | 5 | 15 | 9 | 3 | 0 |
| | 9.0 | 11 | 2 | 3 | 2 | 11 | 3 | 0 | 0 |
| Comparative | 7.2 | 261 | 459 | 1147 | 1403 | 261 | 461 | 1251 | 1655 |
| Example 1 | 7.4 | 245 | 322 | 613 | 1219 | 245 | 328 | 830 | 1329 |
| | 7.6 | 279 | 299 | 582 | 958 | 279 | 311 | 673 | 932 |
| | 7.8 | 228 | 355 | 397 | 628 | 228 | 357 | 474 | 594 |
| | 9.0 | 257 | 251 | 263 | 390 | 257 | 286 | 302 | 356 |
| Comparative | 8.3 | 25 | 11 | 4 | 3 | 25 | 20 | 12 | 4 |
| Example 2 | | | | | | | | | |

TABLE 10

| | | Evaluation Index (25 μm) | | | | | | | |
|---|---|---|---|---|---|---|---|---|---|
| | | Long-Term | | | | Accelerated | | | |
| | | 0 | 1 | 3 | 6 | 0 | 1 | 3 | 6 |
| Example | | day | months | months | months | day | months | months | months |
| Example 1-1 | 7.2 | 15 | 4 | 1 | 1 | 15 | 6 | 1 | 1 |
| | 7.4 | 3 | 1 | 1 | 1 | 3 | 1 | 1 | 1 |
| | 7.6 | 0 | 0 | 0 | 0 | 0 | 0 | 0 | 0 |
| | 7.8 | 3 | 1 | 1 | 0 | 3 | 0 | 0 | 0 |
| | 9.0 | 3 | 1 | 1 | 1 | 3 | 0 | 0 | 0 |
| Example 1-2 | 7.2 | 17 | 3 | 1 | 1 | 17 | 3 | 2 | 1 |
| | 7.4 | 5 | 3 | 1 | 1 | 5 | 0 | 1 | 0 |
| | 7.6 | 1 | 0 | 0 | 0 | 1 | 0 | 0 | 0 |
| | 7.8 | 2 | 1 | 1 | 1 | 2 | 0 | 0 | 0 |
| | 9.0 | 5 | 1 | 1 | 0 | 5 | 1 | 1 | 0 |
| Example 2-1 | 7.2 | 21 | 1 | 1 | 1 | 21 | 2 | 2 | 2 |
| | 7.4 | 11 | 1 | 1 | 1 | 11 | 1 | 0 | 0 |
| | 7.6 | 1 | 0 | 0 | 0 | 1 | 0 | 0 | 0 |
| | 7.8 | 9 | 1 | 0 | 0 | 9 | 1 | 1 | 0 |
| | 9.0 | 7 | 0 | 0 | 0 | 7 | 0 | 0 | 0 |
| Example 2-2 | 7.2 | 20 | 2 | 1 | 1 | 20 | 2 | 2 | 1 |
| | 7.4 | 14 | 2 | 1 | 1 | 14 | 1 | 1 | 1 |
| | 7.6 | 1 | 0 | 0 | 0 | 1 | 0 | 0 | 0 |
| | 7.8 | 3 | 1 | 0 | 0 | 3 | 0 | 0 | 0 |
| | 9.0 | 7 | 1 | 0 | 0 | 7 | 3 | 1 | 0 |
| Example 3 | 7.4 | 4 | 2 | 1 | 1 | 4 | 0 | 0 | 0 |
| | 7.6 | 3 | 2 | 1 | 1 | 3 | 1 | 0 | 0 |
| | 7.8 | 6 | 1 | 0 | 0 | 6 | 1 | 1 | 0 |
| | 9.0 | 1 | 0 | 0 | 0 | 1 | 0 | 0 | 0 |
| Comparative | 7.2 | 21 | 197 | 383 | 632 | 21 | 214 | 478 | 729 |
| Example 1 | 7.4 | 15 | 40 | 173 | 354 | 15 | 48 | 199 | 388 |
| | 7.6 | 4 | 35 | 41 | 187 | 4 | 39 | 42 | 183 |
| | 7.8 | 3 | 31 | 47 | 68 | 3 | 47 | 52 | 60 |
| | 9.0 | 9 | 25 | 34 | 37 | 9 | 21 | 46 | 32 |
| Comparative | 8.3 | 7 | 1 | 1 | 0 | 7 | 3 | 1 | 1 |
| Example 2 | | | | | | | | | |

Instructions above are provided only to describe the present invention, and are not intended to limit the present invention. Descriptions have been made with reference to exemplary embodiments of the present invention, but it will be understood by those ordinary skilled in the art that the present invention may be variously changed and modified without departing from the spirit and field of the present invention, as described in the following scope of claims. Thus, the changes and modifications above are to be all included within the scope of the present invention, as defined in the claims, and the present invention includes the following claims and all the inventions equivalent to the following claims.

What is claimed is:

1. A pharmaceutical composition, wherein the composition comprises deoxycholic acid or pharmaceutically acceptable salts thereof, and pharmaceutically acceptable additives and carriers, and has pH of 6.5 to 8.0 with improved stability.

2. The pharmaceutical composition according to claim 1, wherein the pharmaceutically acceptable additive is one or more selected from the group consisting of a pH-adjusting agent, isotonic agent, preservative, antimicrobial agent and buffer.

3. The pharmaceutical composition according to claim 2, wherein the preservative is one or more selected from the group consisting of benzalkonium chloride, benzaltonium chloride, phenol, cresol, chlorocresol, chlorobutanol and benzyl alcohol.

4. The pharmaceutical composition according to claim 1, wherein the composition is formulated into an aqueous preparation for parenteral administration.

5. The pharmaceutical composition according to claim 1, wherein the composition is for preventing or treating obesity.

6. The pharmaceutical composition according to claim 1, wherein the pharmaceutically acceptable additive comprises one or more selected from the group consisting of benzalkonium chloride and benzaltonium chloride.

7. The pharmaceutical composition according to claim 6, wherein the composition has pH of 7.0 to 7.9.

8. The pharmaceutical composition according to claim 1, wherein the pharmaceutically acceptable additive comprises benzyl alcohol.

9. The pharmaceutical composition according to claim 8, wherein the composition has pH of 7.2 to 7.8.

10. A first pharmaceutical composition, wherein the composition comprises deoxycholic acid or pharmaceutically acceptable salts thereof, and pharmaceutically acceptable additives and carriers and has pH of 8.7 or more with improved stability.

11. A pharmaceutical composition, comprising: the first pharmaceutical composition according to claim 10; and a second pharmaceutical composition comprising one or more selected from the group consisting of vitamin, lidocaine, procaine, L-carnitine, minophylline, caffeine, vitamin C, placenta, pentoxifylline, clostridium botulisum toxin, polydeoxyribonucleotide (PDRN), hyalironic acid, thioctic acid, glutathion, glycyrrhizin, fursultiamine and hyaluronidase.
